(19) United States
(12) Patent Application Publication
(10) Pub. No.: US 2026/0004896 A1
(43) Pub. Date: Jan. 1, 2026

(54) SYSTEM AND METHOD FOR CUSTOMIZED AUTOMATED CLINICAL DIAGNOSTIC CROSSOVER STUDIES

(71) Applicant: Bio-Rad Laboratories, Inc., Hercules, CA (US)

(72) Inventors: John C. Yundt-Pacheco, Fairview, TX (US); Curtis A. Parvin, Garland, TX (US); Abdon Iniguez, Irvine, CA (US)

(73) Assignee: Bio-Rad Laboratories, Inc., Hercules, CA (US)

(21) Appl. No.: 18/881,947

(22) PCT Filed: Jul. 18, 2023
(86) PCT No.: PCT/US2023/070421

§ 371 Date: Jan. 7, 2025

Related U.S. Application Data
(60) Provisional application No. 63/368,993, filed on Jul. 21, 2022.

(51) Int. Cl. G16H 10/40; G16H 50/20

(52) U.S. Cl. CPC G16H 10/40; G16H 50/20

(57) ABSTRACT

Systems and methods for conducting customized automated crossover studies for clinical diagnostic analyzers are disclosed. In disclosed embodiments, an optimal number of samples to run on a new QC material is determined based on historical information of the analyzer instrument and a peer group of similar instruments. Using the determined optimal number of samples, the crossover study is conducted, and the results are reported.

### CROSS-REFERENCE TO RELATED APPLICATIONS

This application is a 371 national phase of, and claims the benefit of, International Application Number PCT/US23/70421, filed Jul. 18, 2023, and U.S. Provisional Application Ser. No. 63/368,993, filed Jul. 21, 2022, the disclosure of which is hereby incorporated herein in its entirety by reference.

### BACKGROUND OF THE INVENTION

The present invention relates generally to clinical diagnostic analyzers, and more particularly to systems and methods for conducting customized automated cross over studies in such analyzers.

Clinical diagnostic laboratories use various quality control schemes to ensure that the clinical diagnostic processes employed and the clinical diagnostic analyzers used to analyze patient specimens, or other test specimens, provide accurate diagnostic results. One common quality control scheme involves testing quality control (QC) materials having known properties using the same analyzers and processes that are used to test patient specimens. Running such quality control tests with material having known properties ensures that the clinical diagnostic analyzers used to perform the test provide accurate results, or provide results within a predetermined range or specification, and likewise ensures that the reagents and processes used in conjunction with the analyzers provide expected results.

While quality control testing using control material having known properties is generally useful, statistical control issues may arise when the control materials must be replenished. Because the control materials have a limited lifetime, and because QC testing using the control material consumes that material, laboratories must regularly deal with obtaining and using new lots of control materials, requiring them to crossover and begin using the new lot of QC material. Crossing over to the new QC material is a substantial undertaking for a laboratory, as the reliability and accuracy of the new control material must be ensured before proceeding with further testing relying on the new control materials. Even though the new lot of QC control material will have similar properties to the previous lot, even small variations between lots will affect the accuracy of the testing, particularly until a sufficient number of tests can be run on the new QC materials. Thus, laboratories must engage in crossover studies to verify the parameters of the new materials before the desired accuracy of the testing can be ensured. Such crossover studies must be done for any change in control materials, because even with control materials that have insert ranges, i.e., assayed control materials, insert ranges are only intended for laboratories to quickly determine if they are in control, they are not intended for use for performance monitoring.

Typical crossover studies involve determining the statistical behavior of a new lot of QC control material, namely, estimating the mean and standard deviation (SD) of the new material. In order to obtain that mean and SD measurement, the general approach for crossover studies has been to evaluate samples and collect data of the new control material over time until sufficient data has been collected to compute the mean and SD from the collected data. Then, once calculated, using and assigning the newly-calculated mean and SD for future control testing using the new quality control materials.

One generally accepted method of making that initial assessment is that described in Statistical Quality Control for Quantitative Measurement Procedures: Principles and Definitions; Approved Guideline-Third Edition, which calls for making a minimum of at least twenty different measurements of control material, for each control level, on separate days. That generally accepted method thus requires collecting at least twenty data points per control level, over a period of twenty days. Thus, for example, for a trilevel control involving thirty separate analytes, ninety separate studies must be conducted with a data point collected for each individual test. That collected data is then used to estimate a mean and SD for the new lot of material. In addition to the time required, such studies incur considerable expense for the laboratory, with each molecular data point collected incurring costs of $200 or more. Such studies are also labor intensive. As there is no standardized system for conducting such crossover studies, most laboratories typically manually process the collected data using a spreadsheet, and manually input the data to calculate the mean and SD of the new control material.

Even after incurring the time, expense, and inefficiency of conducting crossover studies in accordance with the generally recommended procedures, the results of those studies may not have the accuracy desired or required by the laboratory. For example, while twenty data points is sufficient to determine the mean of the new material, collecting that number of data points is not necessary and is thus inefficient, as the mean can be determined by using just ten data points. Thus, the generally recommended crossover study method incurs unnecessary testing and expense with respect to determining the mean. Furthermore, twenty data points is insufficient to determine the SD with a desired level of accuracy, typically eighty data points are required. Using the generally recommended methods thus typically results in estimated SD's having a high error margin, and using a greater number of data points may result in incurring unnecessary time and costs.

Recognizing the above limitations, the industry has suggested an alternative for determining the SD of the new control material based on using only ten data points by incorporating the mean and SD of the old material, using the equation SD_{new}=(MEAN_{new}*CV_{old})/100, where CV_{old}=SD_{old}*100/MEAN_{old}. However, while that alternative determination requires a fewer number of data points, and thus takes less time, the results using that method still incur potential inaccuracies in the mean calculation (see, e.g., C24 Statistical Quality Control for Quantitative Measurement Procedures: Principles and Definitions, 4^{th} Edition).

While a ten data point process is an improvement and saves time, labor, and QC material over the standard twenty point process, it is still a compromise “one size fits all” in that in some instances it may not be necessary to test even ten data points, or in some cases more than ten data points may provide optimal results.

Thus, it is apparent that there remains a need in the art for an improved system and method for conducting automated crossover studies that accounts for variances in test methods, implementation of test methods, analytical concentrations of QC materials, and other systemic variations in the process to increase the accuracy and reduce the time and expense incurred in crossovers studies as compared to generally known methods.

### BRIEF SUMMARY OF THE INVENTION

Systems and methods for conducting automated clinical diagnostic crossover studies are described in PCT Application No. PCT/US20/66424, and systems and methods for virtual crossover studies for clinical diagnostic systems are described in PCT Application No. PCT/US20/66563 which are hereby incorporated herein in their entireties by reference.

The present invention is directed to a system and method for conducting customized automated crossover studies in clinical diagnostic analyzers. In an exemplary embodiment, the system and method of the present invention employs one or more clinical diagnostic analyzers to determine a customized, optimal number of data points/samples of new quality control material to be tested in a crossover study based on historical data from the instrument(s) and its peer group(s), thus minimizing the time, labor, and expense required to implement a crossover study for a new batch of QC material.

In one aspect, a clinical diagnostic analyzer for performing a customized automated crossover study includes a processor, memory, measurement hardware, and an input panel/display. The analyzer prompts a user to load a QC specimen, and to instigate testing and analysis to determine an optimized number of QC samples to test for the new or crossed-over QC material.

In another aspect, an automated method for calculating an optimal number of data points/samples to use in the crossover study includes estimating the historical relationship between a clinical diagnostic analyzer instrument and its peer group, estimating a new control means using the historical relationship and the new control peer data, estimating a confidence factor in the new control means, simulating or calculating the variation of the new control means by sample size, and selecting a sample size that meets desired the performance criteria.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings and claims. In the drawings, like reference numbers indicate identical or functionally similar elements.

### BRIEF DESCRIPTION OF THE DRAWINGS

The present invention will be described in greater detail in the following detailed description of the invention with reference to the accompanying drawings that form a part hereof, in which:

FIG. 1 depicts a block diagram of a clinical diagnostic analyzer system having a plurality of clinical diagnostic analyzers in communication with a server over a network in accordance with an exemplary embodiment of the present invention.

FIG. 2 depicts a block diagram of a single clinical diagnostic analyzer of the system of FIG. 1.

FIG. 3A is a depiction of a first exemplary prompt screen presented by the clinical diagnostic analyzer of FIG. 2.

FIG. 3B is a depiction of a second exemplary prompt screen presented by the clinical diagnostic analyzer of FIG. 2.

FIG. 3C is a depiction of a third exemplary prompt screen presented by the clinical diagnostic analyzer of FIG. 2.

FIG. 3D is a depiction of a fourth exemplary prompt screen presented by the clinical diagnostic analyzer of FIG. 2.

FIG. 4 is a flow diagram of an exemplary method for performing a customized automated crossover study.

FIG. 5 is a flow diagram of an exemplary method for calculating a customized optimal value for a number of samples to use in conducting a customized automated crossover study in accordance with an exemplary embodiment of the present invention.

### DETAILED DESCRIPTION OF EXEMPLARY EMBODIMENTS

Systems and methods for conducting customized automated crossover studies in a clinical diagnostic analyzer, or in groups of clinical diagnostic analyzers, in accordance with exemplary embodiments of the present invention are described herein. While the invention will be described in detail hereinbelow with reference to the depicted exemplary embodiments and alternative embodiments, it should be understood that the invention is not limited to the specific configurations shown and described in these embodiments. Rather, one skilled in the art will appreciate that a variety of configurations may be implemented in accordance with the present invention.

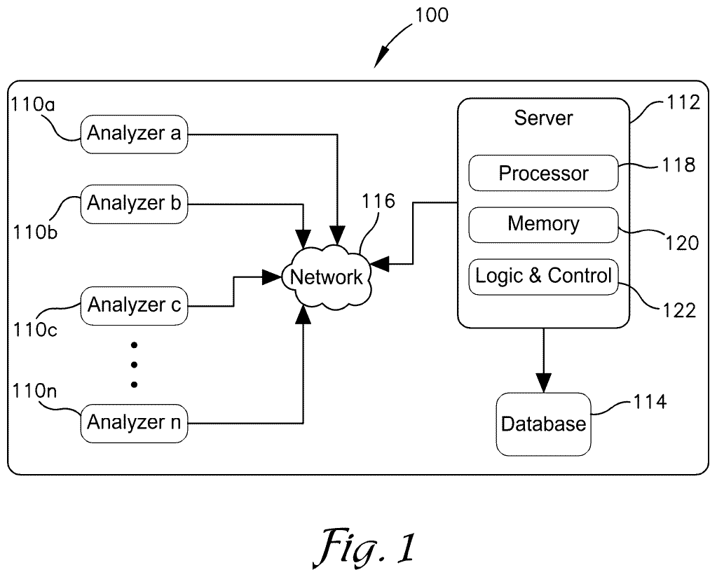

Looking first to FIG. 1, a clinical diagnostic system in accordance with an exemplary embodiment of the present invention is depicted generally by the numeral 100. The system 100 generally includes a plurality of clinical diagnostic analyzers 110a, 110b, 110c, 110n and a server 112 in communication with a database 114. The plurality of clinical diagnostic analyzers 110a, 110b, 110c, 110n are in communication with network 116, which facilitates the transmission of instructions, information, and data between each clinical diagnostic analyzer 110a, 110b, 110c, 110n and the server 112, as well as between each of the clinical diagnostic analyzers 110a, 110b, 110c, 110n and any of the other diagnostic analyzers, or between any combination of clinical diagnostic analyzers and/or the server. As used herein, a clinical diagnostic analyzer may also be referred to as an analyzer or an instrument.

Network 116 may be any local area network (LAN), wide area network (WAN), ad-hoc network, or other network configuration known in the art, or combinations thereof. For example, in the exemplary embodiment depicted in FIG. 1, network 116 may include a LAN allowing communication between the clinical diagnostic analyzers 110a, 110b, 110c, 110n, such as in a single laboratory setting having multiple clinical diagnostic analyzers, and may also include a WAN, such as the Internet or other wide area network, allowing communication between the LAN and the server 112 and/or between the clinical diagnostic analyzers and the server.

It should be understood that the configuration depicted in FIG. 1 is exemplary, and not limiting, and that the invention as described herein may be embodied in a single clinical diagnostic analyzer, in a group of clinical diagnostic analyzers co-located in a single laboratory or facility, and in group of clinical diagnostic analyzers that are geographically dispersed.

For example, multiple systems 100, each comprising one or more clinical diagnostic analyzers and servers may be located in a single laboratory, or in multiple laboratories dispersed across a facility or across the globe, all in communication via a WAN. It should be further understood that the present invention may be embodied in a single clinical diagnostic analyzer, or in a group of clinical diagnostic analyzers in communication with each other over a LAN or WAN, without a server or servers. These and other variations and embodiments will be apparent to those skilled in the art.

Server 112 preferably includes a processor 118, memory 120, and logic and control circuitry 122, all in communication with each other. Server 112 may be any server, server system, computer, or computer system known in the art, preferably configured to communicate instructions and data between the server 112 and the network, and/or to any device connected to the network, and to store and retrieve data and information to and from the database 114. Processor 118 may be any microprocessor, controller, or plurality of such devices known in the art. Processor 118 preferably runs a server operating system such as a Linux based, Windows based, or other server operating system known in the art. Preferably, the processor 118 is configured to control the operation of the server 112 in conjunction with the operating system, allowing the server to communicate with the database 114 and the network 116 and/or with devices connected to the network, such as the clinical diagnostic analyzers 110a, 110b, 110c, 110n. In some embodiments the server may control the operation of the clinical diagnostic analyzers, for example allowing operation of the analyzers during specific time periods, collecting data from the analyzers for storage in the database 114, transferring data to the analyzers for viewing and/or analysis, collecting test data from the analyzers, and providing data, instructions or prompts to the analyzers either individually or in groups.

Memory 120 may be volatile or non-volatile memory and is used to store data and information associated with the operation of the server as well as data for transmission to and from the server. For example, the memory stores the server operating system for execution by the processor 118 and may also store data associated with the clinical diagnostic analyzers 110a, 110b, 110c, 110n in communication with the server 112 over the network 116. In some embodiments the memory 120 on the server may be used as a supplement to, or in place of, the database 114.

The database 114 is preferably used to store control information relating to the operation of the server 112 and the operation and control of the clinical diagnostic analyzers 110a, 110b, 110c, 110n, and may also be used to store data relating to the processing of samples by the clinical diagnostic analyzers. For example, the database may contain instructions or programming for execution by a processor on a clinical diagnostic analyzer, or for execution on the server, or may store data related to the number of samples processed, the frequency of testing, the results of analysis performed on the analyzer, as well as data relating to the samples themselves, such as tracking information, lot numbers, sample size, sample weight, percentage of sample remaining, and the like. Preferably, the database 114 includes non-volatile storage such as hard drives, solid state memory, and combinations thereof.

Logic and control circuitry 122 provides interface circuitry to allow the processor and memory to communicate, and to provide other operational functionality to the server, such as facilitating data communications to and from the network 116.

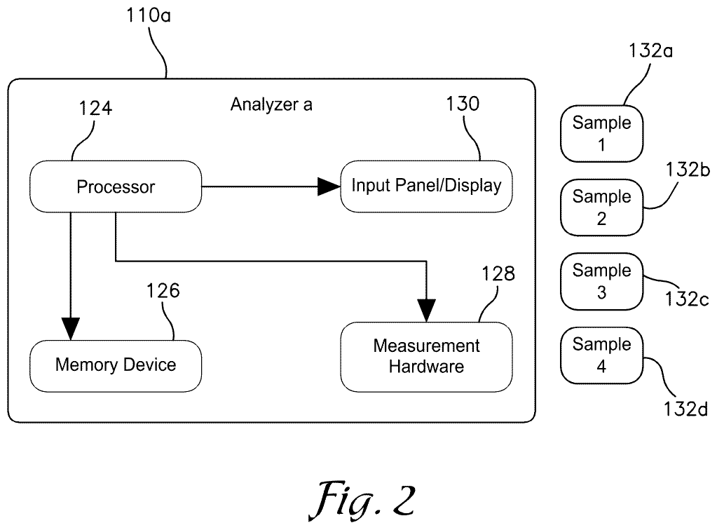

Turning to FIG. 2, a detailed view of a single clinical diagnostic analyzer 110a of the system of FIG. 1 is depicted. Clinical diagnostic analyzer 110a preferably comprises a processor 124, a memory device 126, measurement hardware 128, and an input panel/display 130.

The processor 124 may be any controller, microcontroller, or microprocessor as known in the art, and is in communication with memory device 126 which stores instructions for execution by the processor to control and communicate with the measurement hardware 128 and the input panel/display 130 to cause the clinical diagnostic analyzer to perform desired steps, such as sampling as commanding the measurement hardware to load test specimens or to perform a test on a loaded sample, or instructing or prompting an user to perform specific operations such as replacing a test sample, beginning a test, or viewing collected data. The processor 124 may also execute instructions to receive data from the measurement hardware 128 and to perform one or more analyses on the received data, and to display test results or other information on the input panel/display panel 130.

Measurement hardware 128 preferably includes a sample receptacle configured to receive one or more samples into the analyzer for testing. Preferably, the measurement hardware is configured to receive samples or specimens stored within vials, and most preferably is configured to receive a plurality of vials and to extract samples, i.e., analytes, from any desired specimen vial for testing and analysis. In further embodiments, the measurement hardware 128 may include external turntables, loaders, or other mechanisms to facilitate the loading and unloading of samples to allow samples to be loaded under command of the analyzer.

As depicted in FIG. 2, the measurement hardware is configured to be used with material samples 132a, 132b, 132c, 132d, which may be QC materials, patient test specimens or other specimens as is known in the art. In one embodiment, the material samples are contained in vials which are loaded or inserted into the clinical diagnostic analyzer 110a by an user. The samples may be loaded individually, or in groups, e.g., in a tray that is loaded into the analyzer. In alternative embodiments, the samples may be loaded using an automated loading mechanism, such as a turntable or other mechanism, upon command from the analyzer 110a. Material samples in the form of QC materials are typically provided in lots, with a unique lot number assigned to a lot of samples that are essentially identical as coming from the exact same batch source of material. The analyzer 110a preferably allows information relating to the QC materials to be entered by a user, including statistical information such as a mean or standard deviation for the lot of material. In other embodiments, the information may be obtained over a network or from a server using, for example a QR code on the sample vial or container to uniquely identify the sample or lot.

Input panel/display 130 is in communication with the processor and is operable to present controls to facilitate operation of the analyzer, as well as to present prompts and instructions to an user, and to receive input commands and/or data from the user. The input panel/display 130 is preferably a touch screen having capabilities of displaying text and graphics as well as icons, push buttons, keyboards, and the like to both present data to a user and to receive input from a user of the analyzer. Preferably, the input panel/display 130 includes an audible alert device such as a buzzer or beeper.

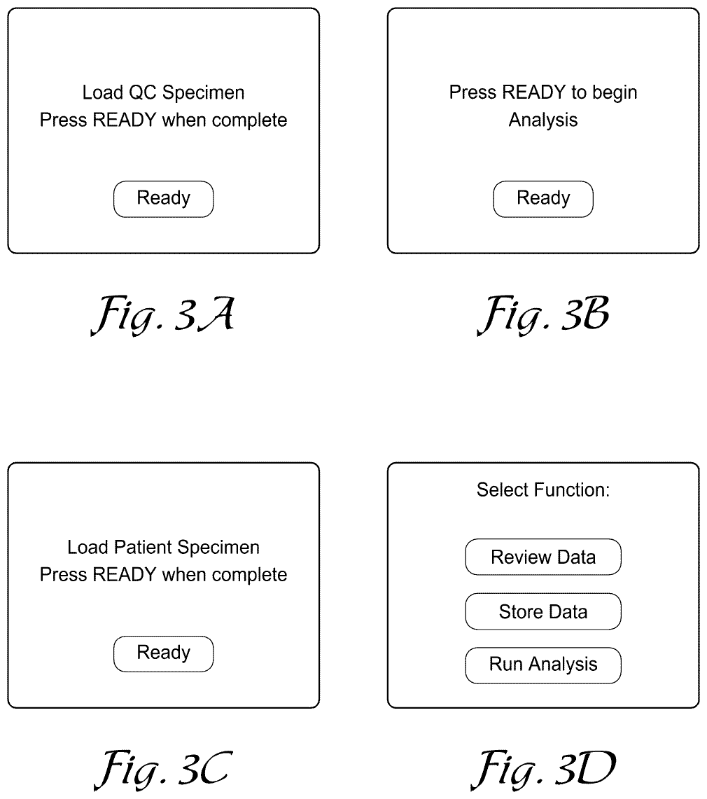

Looking to FIGS. 3A, 3B, 3C, and 3D, for example, the input panel/display may present prompts to a user to load a QC specimen and press a READY button once completed (FIG. 3A), to begin an analysis (FIG. 3B), to load a patient specimen (FIG. 3C) or to select another desired function, such as reviewing data, storing data, or running an analysis (FIG. 3D). It should be understood that the clinical diagnostic analyzer 100a may have multiple programs and functions available, a menu or selection prompt is preferably presented to guide a user through the operation of the analyzer and the selection of desired functions and operations.

Clinical diagnostic analyzer 110a may be any type of analyzer known in the art, such as biochemistry analyzers, hematology analyzers, immune-based analyzers, or any other clinical diagnostic analyzer known in the art. Preferably, analyzer 110a is configured to test quality control materials having known properties to allow users to determine the accuracy of the analyzer and to provide assurance to users that the analyzer is operating within allowable tolerances. Clinical diagnostic analyzer 110a may be configured for use with various quality control materials, whether in liquid or lyophilized form, and may be configured for use in the immunoassay, serum chemistry, immunology, hematology, and other fields.

Looking to FIGS. 1 through 3 in combination, in typical use in performing a test on a patient specimen, the analyzer 110a prompts an user to load a patient specimen as depicted in FIG. 3C, and to perform an analysis as depicted in FIG. 3B once the sample is loaded. Upon completion of the test, the analyzer may prompt the user to store or review the data as seen in FIG. 3D. Similarly, the analyzer may guide a user to begin a test QC material as depicted in FIG. 3A.

It should be understood that the operation of the analyzer 100a may be performed locally, at the analyzer, or that the operation may be coordinated thorough the server 112 when the analyzer is operated in a system 100 as depicted in FIG. 1 It should be further understood that any data may be stored locally on the analyzer 110a, on the server 112 or database 114, and that the data may be made available throughout the system 100 and over the network 116 so that remote servers and analyzers may likewise access the stored data. Similarly, analyses may be run on the analyzer itself, on the server, or may be distributed among multiple analyzers and/or servers.

In embodiments of the invention described herein, the analyses performed on multiple analyzers and the data collected may be analyzed in combination to provide an output or result based on data collected across multiple analyzers.

Known methods of performing crossover studies rely on collection and analysis of data over a relatively long period of time—typically at least twenty days—with data points collected on each of those days and analysis performed once all data has been collected. By contrast, the system and method of the present invention calculates an optimal number of data points/samples necessary to achieve a desired accuracy without requiring that a fixed number of data points be collected over a lengthy and/or fixed period of time. The system and method may further perform a customized automated crossover study in which the optimal number of data points/samples to achieve a desired accuracy is used to perform a QC crossover study using that optimal number of samples.

Thus, the system and method of the present invention provides for an improvement in the field of clinical diagnostic processes by requiring testing only of an optimal number of samples, avoiding cost, labor, and expense that may be incurred when testing using a conventional twenty-day sample and data analysis process, and allows for use of that optimal number of samples in conducting a customized automated crossover study.

With the configuration of the clinical diagnostic analyzers and system set forth, systems and methods for conducting customized automated crossover studies in accordance with embodiments of the present invention will now be described.

As discussed above, in one embodiment, conducting a customized crossover study in accordance with the present invention involves determining an optimal number of samples required to achieve a desired accuracy for a new lot of control material. Because the clinical diagnostic analyzers used to analyze test specimens and patient specimens require calibration and verification, until a laboratory can ascertain the parameters of the new control material, it cannot be certain of the accuracy of the results of analyses performed on actual specimens.

In order to determine an optimal number of samples to run on the new QC material in accordance with an exemplary embodiment of the present invention proceeds generally as follows:
First an estimate of the historical relationship between an instrument and its peer group is determined. Next, new control means are estimated based on the historical relationship between the instrument and its peer group and the new control peer data, and a confidence factor for the new control means is likewise estimated. Then, using the estimated new control means, the confidence factor, and instrument variation, the variation of the new control means by sample size is determined. A customized sample size that meets the desired performance criteria may then be selected for us in conducting the crossover study.

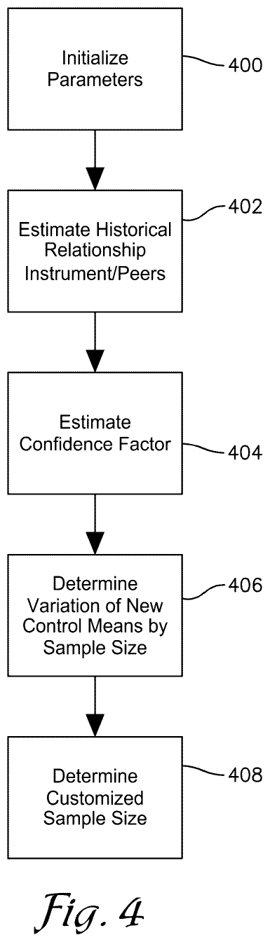

With the general steps of the method of determining a customized sample size for conducting an automated crossover study set forth, the calculations for making that determination will now be described with reference to the flow diagram of FIG. 4. At block 400, parameters for the calculations are defined and initialized as follows:
Lᵢⱼ: is the QC mean for the i^{th} instrument in a peer group on the j^{th} QC lot and concentration level, where:
i=1, . . . . I (the number of instruments in the peer group);
j=1, . . . , Jᵢ (the total number of QC lots and levels for the i^{th} instrument in the peer group); and
Gⱼ is the QC peer group mean for the j^{th} QC lot and level.

With those initial parameters set forth, at block 402, an estimate of the historical relationship between an instrument and its peer group is determined by defining a linear model for the relationship between the individual instrument's QC mean and the peer group mean as:

$${L}_{i j} = {α}_{i} + {β}_{i} {G}_{j} + {e}_{i j}$$

Where αᵢ and βᵢ define the linear relationship between an instrument's QC mean and the peer group mean, and eᵢⱼ are the random deviations in the relationship, and:

$${e}_{ij} ∼ Normal ( 0 , f ( G ) {σ}_{e}^{2} )$$

Next, assuming that αᵢ=0 for all i and f(G)=G², then the model becomes;

Lᵢⱼ=βᵢGⱼ+eᵢⱼ,

$${e}_{ij} ∼ Normal ( 0 , {G}_{j}^{2} {σ}_{e}^{2} ) ,$$

which can be expressed as:

$${L}_{ij} = {β}_{i} {G}_{j} + {G}_{j} {e}_{ij} , {e}_{ij} ∼ Normal ( 0 , {σ}_{e}^{2} ) ,$$

where: βᵢ defines the proportional relationship between the instrument's QC mean and the peer group mean, and the random deviation in the relationship is proportional to the peer group mean.

$$Defining {Y}_{ij} = {L}_{ij} / {G}_{j} , then {Y}_{ij} = {β}_{i} + {e}_{ij} , E ( {Y}_{ij} ) = {β}_{i} , Var ( {Y}_{ij} ) = {σ}_{e}^{2} .$$

Then, the proportional relationship between an instrument and group mean can be estimated as;

$${\hat{β}}_{ι} = \frac{1}{{J}_{i}} {\sum }_{j = 1}^{{J}_{i}} {Y}_{ij} = {\overline{Y}}_{i} .$$

and at block 404, the expected value and variance of [symbol] are;

$$E ( {\hat{β}}_{ι} ) = {L}_{i} / G , Var ( {\hat{β}}_{ι} ) = \frac{{σ}_{e}^{2}}{{J}_{i}}$$

The variance of the proportional relationship between the i^{th} instrument and the peer group mean can thus be estimated as;

$${s}_{i}^{2} = \frac{1}{( {J}_{i} - 1 )} {\sum }_{j = 1}^{{J}_{i}} {( {Y}_{ij} - {\overline{Y}}_{i} . )}^{2}$$

At block 406 an estimate of the variance of the new control means is determined. Assuming the variance of the proportional relationship between an instrument and the peer group mean is the same for all instruments, then the pooled estimate of the variance is:

$${\hat{σ}}_{e}^{2} = {\sum }_{i = 1}^{I} ( {J}_{i} - 1 ) {s}_{i}^{2} / {\sum }_{i = 1}^{I} ( {J}_{i} - 1 )$$

An estimate of an instrument's new lot of QC material requires determining at each concentration level of the new lot based on the historical relationship of the instrument with its peer group and the new QC lot measurements, as follows:

Given [symbol] , an estimate of the historical proportional relationship between the i^{th} lab's QC lot means and it's peer group means, G_{new,l} the peer group mean for a new QC lot and concentration level l, and {x_{ilq}}, q=1, . . . , Qₗ, representing Qₗ new QC lot measurements for the QC concentration level l on the i^{th} instrument.

Define E(x_{ilq})=μ_{il,new},

$$Var ( {x}_{ilq} ) = {σ}_{il , new}^{2} ,$$

as the mean and variance of the new QC lot and concentration level measured on the i^{th} instrument in the peer group. Assume a constant coefficient of variation, CVᵢₗ, for an instrument across QC lots for the lot concentration level, l.

Define the initial estimate of the new QC lot/level for instrument i as \hat{L}_{0,i}=G_{new,l}\hat{β}ₗ.

Define kᵢₗ=SD(\hat{L}_{0,i})/σ_{il,new}. An estimate for kᵢₗ can be derived as follows.

$$Var ( {\hat{L}}_{0 , i} ) = {G}_{new , l}^{2} Var ( {\hat{β}}_{i} ) + {G}_{new , l}^{2} {σ}_{e}^{2}$$

$$SD ( {\hat{L}}_{0 , i} ) = {G}_{new , l} \sqrt{{σ}_{e}^{2} ( 1 + 1 / {J}_{i} )}$$

$${σ}_{il , new} = {CV}_{il} E ( {x}_{il , new} ) = {CV}_{il} {G}_{new , l} E ( {L}_{old} / {G}_{old} ) = {CV}_{il} {G}_{new , l} {β}_{i}$$

Thus, at block 408, a customized sample size for the instrument can be determined using the following equations:

$${k}_{il} = \frac{\sqrt{{σ}_{e}^{2} ( 1 + 1 / {J}_{i} )}}{{β}_{i} {CV}_{il}}$$

and an estimate for kᵢₗ is as follows:

$${\hat{k}}_{il} = \frac{\sqrt{{\hat{σ}}_{e}^{2} ( 1 + 1 / {J}_{i} )} / {\hat{β}}_{i}}{{CV}_{il}}$$

With these equations set forth, the customized sample replicate sizy may be determined using a simulation to determine how many sample replicates are needed to keep the increase in the probability of false rejection due to the uncertainty of the mean below a specified threshold, such as 0.015.

First, the probability of false rejection is computed using:

$$Φ = normal cumulative distribution function ( Z , mean , SD ) ; and$$

$$Pfr ( mean , SD ) = 1 - Φ ( 3 , mean , SD ) - Φ ( - 3 , mean , SD )$$

The baseline probability of false rejection (Pfr0) is computed from the instrument mean and SD, as:

$$r 0 = a random selection from a normal distribution N ( 0 , 1 )$$

$$r 1 = a random selection from a normal distribution N ( 0 , 1 )$$

$$w 0 = 1 / ( 1 + q * k \hat{} 2 )$$

$$minst = instrument mean$$

$$sinst = CV * minst$$

$$simulated mean ( smean ) = minst + w 0 * r 0 * k * sinst + ( ( 1 - w 0 ) * r 1 * sinst ) / √ q$$

$$ssd = smean * CV$$

$$simulated Pfr = Pfr ( smean , ssd )$$

To simulate the probability of a false rejection for a mean computed from q samples for a given CV and k:

$$Pfr 0 = Pfr ( instrument mean , intrument SD )$$

Then, starting with a single sample (q=1), simulate 10,000 Pfr's.

Let sPfr=the 80th percentile of the 10,000 simulated Pfr's

If sPfr≤Pfr0+threshold, then q samples are required.

If sPfr>Pfr0+threshold, then increase q by 1 and compute a new sPfr.

Continue increasing q until sPfr≤Pfr0+threshold or q=10.

The q (number of sample replicates) that satisfies sPfr≤Pfr0+threshold is the number of samples required for the customized virtual crossover study.

It should be understood that by collecting historical data for the instrument, peer group, and calculations as set forth above, the above-described approach may be used to estimate k across a ranger of analytes and peer groups. It should be further understood that the distribution of the k estimates may be used to determine the applicability and effectiveness of the estimation in reducing the sample size requirement for a new QC lot and for establishing new QC lot means for a given instrument.

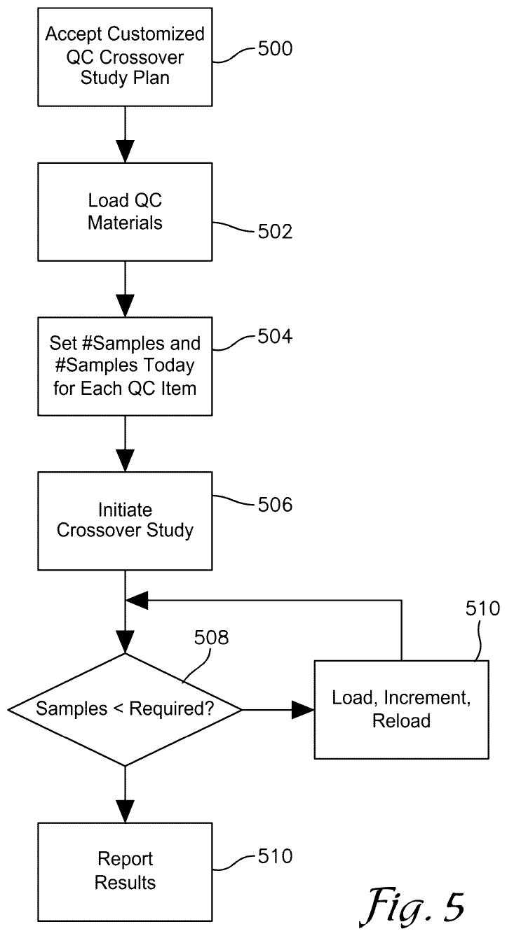

With the method for determining a customized optimal value for use with the new lot of QC material for the instrument, an exemplary method of performing a customized automated crossover study on the instrument in accordance with an exemplary embodiment of the present invention will now be described with reference to FIG. 5.

Looking to the flow diagram of FIG. 5, a customized automated crossover study begins at block 500, where a customized QC crossover study plan containing the number of samples and samples per day required for each QC item being evaluated is loaded or entered into an instrument and/or server for execution on an instrument.

At block 502 the required QC materials are loaded into the analyzer/instrument. As described above, the materials may be manually loaded, or in some cases the instrument and/or laboratory may have an automated system for loading materials.

At block 504, the number of samples to be tested and number of samples to be tested today are initialized.

At block 506, the automated crossover study is initiated using the customized optimal number of samples as previously determined.

At block 508, the testing continues until the desired number of samples have been tested. If the number of samples tested has not been achieved then, at block 510 a QC sample is loaded and tested, and the count of the number of samples already tested is incremented. The process of testing and loading samples is repeated until the desired number of samples have been tested.

At block 512, once the optimal number of samples have been tested, then the crossover study is complete and the results are reported, the results may be analyzed and stored, and alerts may be generated based on the results.

As can be seen, the system and method of the present invention as described herein with respect to exemplary embodiments provides for determining an optimal number of samples/data points for conducting a crossover study and provides cost, time, and labor savings over crossover studies employing a fixed number of samples as is known in the prior art.

While the present invention has been described and illustrated hereinabove with reference to various exemplary embodiments, it should be understood that various modifications could be made to these embodiments without departing from the scope of the invention. Therefore, the invention is not to be limited to the exemplary embodiments described and illustrated hereinabove, except insofar as such limitations are included in the following claims.

What is claimed and desired to be secured by Letters Patent is as follows:

1. A clinical diagnostic analyzer for conducting customized automated crossover studies, comprising:
a processor;
measurement hardware in communication with the processor and configured to measure properties of a specimen;
a memory device having stored thereon executable instructions that, when executed by the processor, cause the clinical diagnostic analyzer to perform operations comprising:
determining a customized number of samples required to achieve a desired accuracy for a new lot of quality control material;
analyzing, over a period of time, a number of QC specimens corresponding to the customized number of samples;
storing a result for each of the analyzed QC specimens; and
loading and testing an analyte from a patient specimen using the stored results.

2. The clinical diagnostic analyzer of claim 1, wherein determining a customized number of samples required to achieve a desired accuracy comprises:
estimating a historical relationship between the clinical diagnostic analyzer and a peer group.

3. The clinical diagnostic analyzer of claim 2, further comprising estimating new control means based on the estimated historical relationship.

4. The clinical diagnostic analyzer of claim 3, further comprising:
calculating a confidence factor based on the estimated historical relationship.

5. The clinical diagnostic analyzer of claim 3, further comprising:
determining a variation of the new control means.

6. The clinical diagnostic analyzer of claim 1, further comprising an input panel and display operable to present information and data from the processor to a user and to accept input and selections from a user.

7. The clinical diagnostic analyzer of claim 6, wherein the memory device includes instructions, that when executed, further cause the clinical diagnostic analyzer to perform operations comprising:
presenting a prompt on the input panel and display to a user to load a QC sample into the measurement hardware; and
accept an input from the user indicating that the analyte has been loaded.

8. A system for conducting customized automated crossover studies, comprising:
a server comprising a processor, a memory and a database;
a plurality of clinical diagnostic analyzers in communication with the server, wherein each of the plurality of clinical diagnostic analyzers comprises:
a processor;
measurement hardware in communication with the processor and configured to measure properties of a specimen;
a memory device having stored thereon executable instructions that, when executed by the processor, cause the clinical diagnostic analyzer to perform operations comprising:
loading a specimen from a new lot of quality control material into the measurement hardware;
wherein the memory of the server has stored thereon executable instructions that, when executed by the server processor, cause the server to perform operations comprising
determining a customized number of samples required to achieve a desired accuracy for a new lot of quality control material;
analyzing, over a period of time, a number of QC specimens corresponding to the customized number of samples;
storing a result for each of the analyzed QC specimens; and
loading and testing an analyte from a patient specimen using the stored results.

9. The system of claim 8, wherein the server memory includes instructions, that when executed, further cause the server to perform operations comprising:
estimating a historical relationship between the clinical diagnostic analyzer and a peer group.

10. The system of claim 8, wherein the server memory includes instructions, that when executed, further cause the server to perform operations comprising:
prompting a user of one or more of the plurality of clinical diagnostic analyzers to load and test an analyte from a patient specimen.

11. The system of claim 8, wherein each of the plurality of clinical diagnostic analyzers comprises an input panel and display, and wherein the server memory includes instructions, that when executed, further cause the server to perform operations comprising:
transmitting an instruction to at least one of the plurality of clinical diagnostic analyzers to present a prompt on the input panel and display to a user to load an analyte into the measurement hardware; and
accept an input from the user of the at least one of the plurality of clinical diagnostic analyzers indicating that the analyte has been loaded.

12. A method for conducting customized automated crossover studies, comprising:
determining a customized number of samples required to achieve a desired accuracy for a new lot of quality control material;
loading a specimen from a new lot of quality control material into measurement hardware of a clinical diagnostic analyzer;
analyzing the specimen to obtain a data value corresponding to an attribute of the specimen;
obtaining and storing data values from a number of QC samples corresponding to the customized number of samples;
storing the obtained data values for each specimen;
loading and testing an analyte from a patient specimen using the stored mean and standard deviation.

13. The method of claim 12, further comprising:
estimating a historical relationship between the clinical diagnostic analyzer and a peer group.

14. The method of claim 12, further comprising:
estimating new control means based on the estimated historical relationship.

15. The method of claim 12, further comprising:
calculating a confidence factor based on the estimated historical relationship.

16. A clinical diagnostic analyzer for conducting crossover studies, comprising:
a processor;
measurement hardware in communication with the processor and configured to measure properties of a specimen;
a memory device having stored thereon executable instructions that, when executed by the processor, cause the clinical diagnostic analyzer to perform operations comprising:
determining an optimal number of samples required to achieve a desired accuracy for a lot of quality control material;
analyzing, over a period of time, a number of QC specimens corresponding to the optimal number of samples; and
loading and testing an analyte from a patient specimen.

17. The clinical diagnostic analyzer of claim 16, wherein determining an optimal number of samples required to achieve a desired accuracy comprises:
estimating a historical relationship between the clinical diagnostic analyzer and a peer group.

18. The clinical diagnostic analyzer of claim 17, further comprising estimating new control means based on the estimated historical relationship.

19. The clinical diagnostic analyzer of claim 18, further comprising:
calculating a confidence factor based on the estimated historical relationship.

20. The clinical diagnostic analyzer of claim 18, further comprising:
determining a variation of the new control means.
